(19) United States
(12) Patent Application Publication
(10) Pub. No.: US 2024/0350110 A1
(43) Pub. Date: Oct. 24, 2024

(54) METHOD FOR ASCERTAINING AN OPERATING PARAMETER AND MEDICAL IMAGING FACILITY

(71) Applicant: Siemens Healthineers AG, Forchheim (DE)

(72) Inventors: Patrick KUEHNLEIN, Kalchreuth (DE); Helmut KROPFELD, Forchheim (DE)

(73) Assignee: Siemens Healthineers AG, Forchheim (DE)

(21) Appl. No.: 18/643,283

(22) Filed: Apr. 23, 2024

(30) Foreign Application Priority Data
Apr. 24, 2023 (DE) 10 2023 203 737.8

(51) Int. Cl. A61B 6/58; A61B 6/00; A61B 6/42

(52) U.S. Cl. CPC A61B 6/585; A61B 6/4241; A61B 6/54

(57) ABSTRACT

A method for ascertaining an operating parameter of a medical imaging device, the method comprising: providing a measurement dataset by operation of the medical imaging device; and ascertaining the operating parameter as a function of the measurement dataset by a processing algorithm. The processing algorithm includes a sub-algorithm, which provides an intermediate result, wherein the intermediate result depends on a first partial dataset of the measurement dataset, wherein the operating parameter is ascertained as a function of the intermediate result and a second partial dataset of the measurement dataset, and wherein calculation of the intermediate result by the sub-algorithm and the capture of the second partial dataset by the medical imaging device at least partially overlap time-wise.

### CROSS-REFERENCE TO RELATED APPLICATION(S)

The present application claims priority under 35 U.S.C. § 119 to German Patent Application No. 10 2023 203 737.8, filed Apr. 24, 2023, the entire contents of which is incorporated herein by reference.

### FIELD

One or more example embodiments of the present invention relate to a method for ascertaining an operating parameter of a medical imaging facility (or device), wherein a measurement dataset is provided by operation of the medical imaging facility, wherein the operating parameter is ascertained as a function of the measurement dataset by a processing algorithm. In addition, one or more example embodiments of the present invention relate to a medical imaging facility and a non-transitory computer-readable storage medium.

### BACKGROUND

In the field of medical imaging, in particular imaging on the basis of radiation detection, for improved comparability of image data of different medical imaging facilities and for avoiding imaging artifacts, it is expedient in the course of production of the medical imaging facility (or device) or a service to carry out a calibration for determining suitable operating parameters or for validating the imaging facility to check whether corresponding operating parameters lie in an admissible tolerance range.

Determination of corresponding operating parameters requires time-consuming measurement and calculation processes and, for example for a modern computed tomography facility, in particular when single photon detection is used, can occupy more than 10 hours in the course of final inspection after production. Tests or parameter determinations in the course of a normal service, which is regularly required, can be relatively time-consuming and require, for example, 30 minutes or more. Typically, the addition of new functionalities results in further operating parameters having to be ascertained, moreover, whereby the times mentioned above can be lengthened even more.

This results in high costs, in particular since during a calibration or validation in the course of production the time until the finished medical imaging facility is provided is appreciably delayed and since with a calibration or validation of an apparatus already being used, the apparatus is not operational for a relatively long time.

### SUMMARY

One or more example embodiments of the present invention are based on at least the object of reducing the required amount of time for calibration or validation of a medical imaging facility (also referred to as a medical imaging device).

At least this object is achieved by a method of the type described in the introduction, wherein the processing algorithm comprises a sub-algorithm which provides an intermediate result, wherein the intermediate result depends solely on a first partial dataset of the measurement dataset, wherein the operating parameter is ascertained as a function of the intermediate result and a second partial dataset of the measurement dataset, wherein the calculation of the intermediate result by the sub-algorithm and the capture of the second partial dataset by the medical imaging facility at least partially overlap time-wise.

Until now, when ascertaining operating parameters of a medical imaging facility, the measurement dataset was only processed after capture of the measurement data was complete. Since the necessary data processing can be quite laborious, and in the examples mentioned above is responsible for approx. 60% of the time required, the total time required can be significantly reduced by at least partially shifting the data processing activities into the time interval used for ascertaining the measurement data. Working hours of production or maintenance staff, the time until the finished product is provided and outage times can be reduced hereby and thus a significant cost reduction achieved. Preferably, the inventive method is computer-implemented.

Primarily the ascertainment of at least one operating parameter, which is to be used later in the course of medical data capture, will be discussed below. An operating parameter ascertained in the manner discussed can also be used for validating the produced or maintained imaging facility, however, with it being possible to check, for example, whether the ascertained operating parameter lies within a specified tolerance interval. If this is not the case, then, for example, a change to hardware components of the imaging facility can be necessary.

Preferably, a plurality of operating parameters can be ascertained. A shared intermediate result can be used for ascertaining the different operating parameters. Alternatively, at least for some of the operating parameters to be ascertained, a separate intermediate result can also be ascertained and/or the intermediate result explained above can be used only for some of the operating parameters, for example when, for one or more operating parameter(s) to be ascertained, no calculations should be carried out at the same time as capture of measurement data owing to a high time criticality. Different examples of ascertainable operating parameters, for which computing steps can be carried out at the same time as data capture, will be discussed later.

A partial dataset can also be processed in the context of embodiments of the present invention for other pairs of partial datasets, at the same time as or overlapping time-wise, for processing a different partial dataset, as is explained above by way of example for the first and second partial datasets. By way of example, on completion of capture of the second partial dataset, this dataset can be processed further, in its own right or in conjunction with the first partial dataset or with processing data dependent on the first partial dataset or intermediate results, in order to provide a further intermediate result while simultaneously or overlapping time-wise a third partial dataset is captured, etc.

Since the measurement dataset is used in the inventive method, in particular, only to ascertain one or a few operating parameter(s), in a preferred embodiment, in contrast to customary methods for medical imaging, it is possible to not store the measurement dataset itself and/or data other than the ascertained operating parameter or the ascertained operating parameters.

The intermediate result or intermediate results ascertained for different partial datasets can be, in particular, preprocessed raw data. Preferably, no reconstructed image data is provided in the course of processing during the capture of measurement data. By way of example, with three-dimensional X-ray imaging, the intermediate result can describe two-dimensional image data, statistical properties of two-dimensional image data or the like or not describe a reconstructed three-dimensional image dataset at least.

The sub-algorithm can be implemented by a first sub-facility, wherein the first sub-facility transfers the intermediate result and/or a processing result ascertained as a function of the intermediate result to a second sub-facility by which the operating parameter is ascertained. The processing algorithm can thus be carried out in a distributed manner by the first and second sub-facilities or, apart from the sub-algorithm, can comprise at least one further sub-algorithm which is carried out by the second sub-facility.

The first sub-facility is preferably arranged close to the imaging facility or its detector or is integrated in the imaging facility itself or is attached to it at least with a high bandwidth. The first sub-facility is optimized, in particular, for simultaneous implementation of calculations. By way of example, calculations for processing the measurement dataset or at least the first partial dataset can occur there on a graphics card or, generally, by SIMD processing (“single instruction multiple data”). Correspondingly configured processing facilities or first sub-facilities are frequently used in the course of conventional medical imaging anyway for preparing the raw data or for image reconstruction and are very suitable for implementing the sub-algorithm in the inventive method. The inventive method can therefore be implemented with little effort by slight modification of customary medical imaging facilities, which can occur, in particular, by way of a pure change in software. The first sub-facility can, in particular, also control components used for capture of the measurement dataset, therefore, for example, an X-ray tube, detectors, an actuating element for adjusting these components, etc.

For example, a desktop PC, a server or any other data processing facility typically decoupled from the medical imaging facility and not time-critically involved in its control, can be used as the second sub-facility. The second sub-facility can thus execute relatively flexibly appropriate software whose change has no effect, in particular, on time-critical measuring tasks of the medical imaging facility. The ascertainment of the operating parameter can be implemented hereby, for example, as a separate subprogram of medical imaging software or also as software which can be started separately. Interventions in the programming of the first sub-facility, which is sometimes relevant to measuring, can thus be minimized.

The first and second sub-facilities can communicate via any communications interface, for example via a bus or a network. By way of example, communication can be via a CAN bus or Ethernet, but also via any other wired or wireless interfaces. Preferably, the measurement dataset, or at least the first partial dataset, is not sent to the second sub-facility if solely operating parameters are to be ascertained.

The transfer of the intermediate result or of the processing result to the second sub-facility and the capture of the second partial dataset by the medical imaging facility can at least partially overlap time-wise. A transfer as early as during capture of the second partial dataset has two advantages. Firstly, time is immediately saved since, owing to the potentially large volumes of data to be transferred, the data transfer, if it were to take place only after completion of capture, would additionally require a significant period of time. Secondly, further processing of the intermediate result or of the processing result on the second sub-facility is potentially already made possible before capture of the second partial dataset is complete.

After its ascertainment the operating parameter can be transferred by the second sub-facility to the first sub-facility and be stored there in order to use the ascertained operating parameter during subsequent imaging by the medical imaging facility for processing captured raw data and/or for controlling at least one component of the medical imaging facility by the first sub-facility. The operating parameter can be stored, in particular, in such a way that the value of the operating parameter is retained without current, for example by using a flash memory. In addition or alternatively, the operating parameter can be stored in a volatile memory, however, for example if it is transferred as part of the initialization of the first sub-facility on starting the medical imaging facility or even only with an image capture command transferred to the first sub-facility.

Use of the operating parameter in the first sub-facility can be particularly advantageous if the operating parameter is used relatively early in the processing of the measurement data, for example as a scaling factor, as part of a lookup table or for specifying threshold values in the case of single photon detection. In the context of control, the operating parameter can be used, for example, to control operating voltages or activation times of an X-ray tube.

In an advantageous embodiment of the inventive method, an ascertainment instruction sent from the second sub-facility to the first sub-facility triggers capture of the measurement dataset as well as at least the execution of the sub-algorithm by the first sub-facility. The instant of execution of the sub-algorithm can be determined immediately hereby on the part of the first sub-facility, whereby a loss of time due to communication between the sub-facilities in the course of simultaneously running capture and processing can be avoided. In addition, in this case the first sub-facility does not have to react, or at least not in a time-critical manner, to incoming communication, so use of the processing resources that are available in the first sub-facility can be improved.

In particular, the ascertainment instruction can be transferred via the same interface via which imaging instructions, which serve for ascertaining medical image data, are also transferred.

In addition or alternatively, it is also possible that capture in a different way of the measurement dataset and/or the execution of mutually different sub-algorithms can be triggered by different ascertainment instructions or by a different parameterization of the ascertainment instruction, and this can be used, for example, to send different ascertainment instructions for ascertaining different operating parameters or to parameterize the ascertainment instructions for them in different ways. The ascertainment of different operating parameters on the part of the second sub-facility can be implemented hereby in that different or differently parameterized ascertainment instructions are sent to the first sub-facility for the different operating parameters and/or in that data received by the first sub-facility is processed differently.

Depending on the operating parameter to be ascertained, the ascertainment instruction can specify whether an inventive parallelization of measurement data capture and data processing should take place or whether the customary procedure, data processing only after complete capture of the measurement dataset therefore, should be reverted to. By way of example, the actuation of components of the medical imaging facility can be particularly time-critical for the ascertainment of particular operating parameters, so it can be advantageous to avoid additional loading of the first sub-facility.

It is possible, in particular, that no further ascertainment instructions and/or imaging instructions can be received and/or processed on the part of the first sub-facility before all measuring and processing procedures triggered by a preceding received ascertainment instruction are complete or the operating parameter, for whose ascertainment the ascertainment instruction was sent, is ascertained. In principle it is also possible to enable simultaneous processing of a plurality of ascertainment instructions and/or imaging instructions although this would result in much greater complexity in the implementation of the first sub-facility, which, as a rule, should be avoided.

The ascertainment instruction can request provision of the intermediate result or the processing result for ascertaining the operating parameter and optionally also of reconstructed image data. If an ascertainment instruction requests both of these, simultaneous processing and capture of measurement data can take place, in particular, only for the reconstruction of the image data since it frequently should be immediately output to a user and delays in this regard should be avoided therefore. The inventive overlapping of the ascertainment of the intermediate result and capture of the second image dataset can thus optionally take place only when the ascertainment instruction does not demand provision of reconstructed image data. With a sufficiently powerful first sub-facility, a parallelization can also take place both for the reconstruction of the image data and for ascertainment of the intermediate result in order to ascertain the operating parameter, however. In principle it would also be possible to proceed inventively in the case of a request for the intermediate result or the processing result and the reconstructed image data, but to carry out ascertainment of the reconstructed image data only after the end of the capture of measurement data.

The measurement dataset can comprise measurement data for a plurality of capture angles, wherein the first partial dataset comprises the measurement data for one or more of the capture angle(s). In particular, the first partial dataset does not comprise the measurement data for all of the capture angles. The measurement data for all remaining capture angles or for at least some of the remaining capture angles can form the second partial dataset or be part of the second partial dataset.

Measurement data for different capture angles is captured, for example, in computed tomography and in molecular imaging. In the simplest case, one separate partial dataset can be captured per capture angle and during the capture of measurement data for a capture angle or of the assigned partial dataset, the partial dataset for the immediately preceding captured capture angle and/or for at least one capture angle captured even earlier can be processed in order to ascertain the intermediate result.

In the course of development of embodiments of the present invention it has been recognized that for determining a large number of operating parameters, projection images or other measurement data are/is very suitable for a respective capture angle in order to ascertain intermediate results, for example a noise component or mean intensity values. It can also be expedient, however, if the respective partial dataset comprises measurement data for a respective angle segment, in particular for a group of capture angles therefore, for which successive measurement data was captured. In some cases it can also be expedient in this connection if the angle segment comprises 360° in order, for example, to evaluate a single rotation of a detector about an axis of rotation in the course of a measuring procedure which comprises a plurality of these rotations.

The sub-algorithm can comprise a summation and/or a logarithmizing of measurement data of the partial dataset or preprocessed data ascertained from this and/or an assignment of value to the measurement data or to the preprocessed data with the aid of a lookup table and/or a statistical evaluation of the measurement data or the preprocessed data, in particular an averaging and/or the ascertainment of a standard deviation, and/or the ascertainment of a noise figure relating to the noise component in the measurement data. It has been recognized that these operations can be carried out for ascertaining a large number of relevant operating parameters to be ascertained before the entire measurement dataset is known, for example for a single projection image or also for individual image lines or measuring points.

On the one hand, a respective operating parameter can be ascertained by way of a plurality of processing algorithms, with the plurality of processing algorithms comprising at least one parallelizable sub-algorithm respectively. On the other hand, the processing algorithm can comprise a plurality of parallelizable sub-algorithms. In both cases the parallelizable sub-algorithms can process the first partial dataset and/or a further partial dataset respectively, which is captured before the second partial dataset time-wise, in order to ascertain a respective intermediate result, with the calculations of these intermediate results at least partially overlapping one another time-wise and the capture of the second partial dataset by the medical imaging facility due to the respective parallelizable sub-algorithm.

A further reduction in the time necessary for the calibration or validation of the medical imaging facility can be achieved by this further parallelization with sufficient computing power of the processing facility, in particular the first sub-facility. In one exemplary embodiment implemented in the course of development of embodiments of the present invention, up to four sub-algorithms were executed at the same time as measurement data capture.

A measure of the sensitivity of detector elements of the medical imaging facility and/or at least one threshold value for single photon detection and/or at least one scaling factor used by the medical imaging facility in the course of imaging and/or at least one entry of a lookup table used in the course of imaging and/or at least one parameter of a filter function used in the course of imaging can be ascertained as an operating parameter. An ascertainment of said operating parameters in the course of a calibration of a medical imaging facility is known per se. The inventive procedure, in which at least the ascertainment of the intermediate result takes place at the same time as some of the capture of measurement data, can significantly reduce the time required for this, however.

An X-ray facility, in particular a computed tomography facility, a facility for molecular imaging or a magnetic resonance tomograph can be used as the medical imaging facility. Examples of molecular imaging are positron emission tomography (PET) and SPECT (single photon emission computed tomography). Use of the inventive method is particularly advantageous for photon-counting X-ray facilities since their calibration is particularly time-consuming.

In addition to the inventive method, an embodiment of the present invention relates to a medical imaging facility, comprising at least one detector for capturing a measurement dataset and a processing facility, wherein the processing facility is configured for carrying out the inventive method. The features explained in relation to the inventive method can be transferred with the advantages described there to the inventive medical imaging facility, and vice versa.

The processing facility can comprise a first sub-facility and a second sub-facility, wherein the first sub-facility is configured to implement the sub-algorithm and to transfer the intermediate result and/or a processing result ascertained as a function of the intermediate result to the second sub-facility, wherein the second sub-facility is configured for ascertaining the operating parameter as a function of the intermediate result and/or the processing result. Details and advantages of this procedure have already been explained above with reference to the inventive method.

In addition, an embodiment of the present invention relates to a non-transitory computer program product including a computer program, comprising instructions which are configured to carry out the inventive method and/or to implement a first and/or second sub-facility of the inventive medical imaging facility when executed on a data processing facility.

An embodiment of the present invention relates, moreover, to a non-transitory computer-readable storage medium or data carrier, which comprises a computer program. The data carrier is, in particular, computer-readable or generally electronically- or machine-readable. When the first and second sub-facilities are used to embody the processing facility, the data carrier can comprise instructions for both sub-facilities. This is expedient, in particular, if the second sub-facility transfers the instructions intended for the first sub-facility, for example in the course of a software installation, the initialization of the medical imaging facility during a starting process or together with an ascertainment instruction, to this first sub-facility. It can also be expedient, however, to use separate data carriers for the programs of the first and second sub-facility.

The features explained above in relation to the inventive method or in relation to the inventive medical imaging facility, can be transferred with the advantages mentioned there to the inventive computer program or the inventive data carrier, and vice versa.

### BRIEF DESCRIPTION OF THE DRAWINGS

Further advantages and details of the present invention can be found in the following exemplary embodiments and the associated drawings. In the drawings, schematically:

FIG. 1 shows a flowchart of an exemplary embodiment of the inventive method, and

FIG. 2 shows an exemplary embodiment of an inventive medical imaging facility.

### DETAILED DESCRIPTION

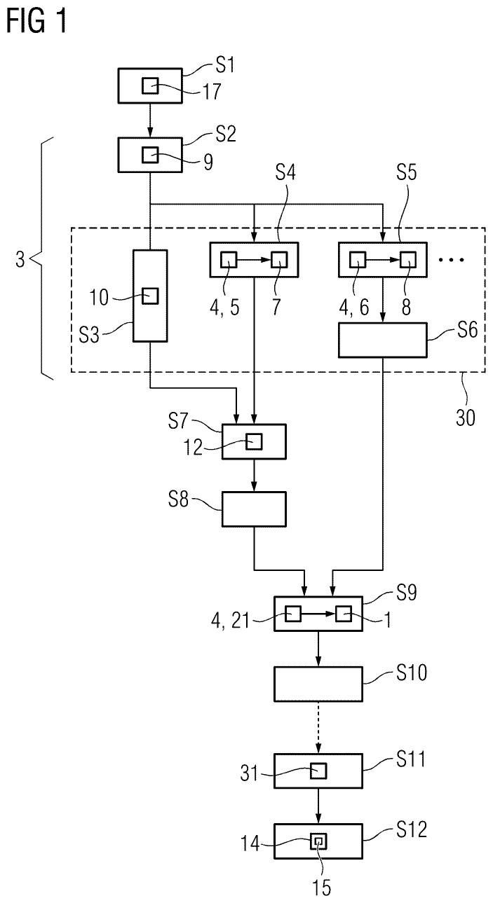

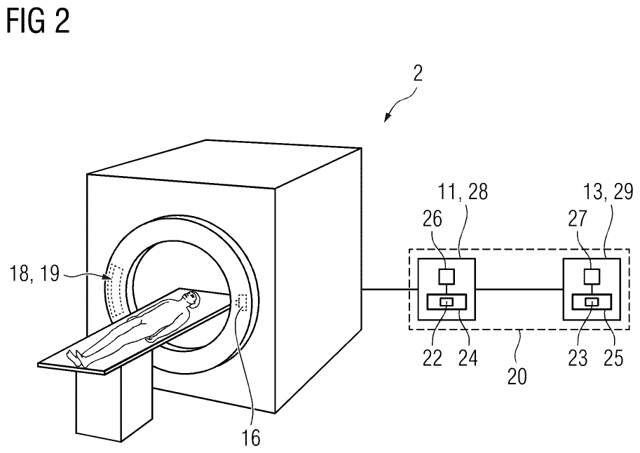

FIG. 1 shows a flowchart of a method for ascertaining an operating parameter 1 of a medical imaging facility 2. One example of an imaging facility 2, for which the method can be used or which is suitable for implementing the method, is shown in FIG. 2 which will also be used for the following explanation of the method for a better understanding of the method sequence.

Following production and sometimes also following a service of medical imaging facilities it can be necessary to calibrate them, to determine particular operating parameters therefore. Such operating parameters can be, for the computed tomograph shown in FIG. 2 as an example of an imaging facility 2, for example, a measure of the sensitivity of detector elements 18 of the detector 19, threshold value for single photon detection, for example for detection of photons with different photon energies, scaling factors used in the course of imaging or entries in a lookup table, for example for contrast optimization, and the like. In addition or alternatively, the operating parameters can also relate to the control of components 16 of the imaging facility 2, for example the X-ray tube.

Algorithms for ascertaining appropriate operating parameters are known per se and are used, for example, in the course of production and maintenance of common facilities for X-ray imaging and for molecular imaging. As already mentioned in the introduction, with customary approaches for ascertaining appropriate operating parameters, it is disadvantageous, however, that these are very time-consuming since firstly all relevant measurement data is fully captured and only then is this measurement data processed.

The method represented in FIG. 1 differs from this procedure primarily in that the processing algorithm 4, which ascertains the operating parameter as a function of measurement dataset 3, is divided into a plurality of sub-algorithms 5, 6, 21, with at least a first one of these sub-algorithms, in the example the sub-algorithms 5, 6, already being carried out before ascertainment of the measurement dataset 3 has been completed.

This is achieved in that firstly only a first partial dataset 9 of the measurement dataset 3 is captured, after which intermediate results 7, 8, which depend on this partial dataset 9, are ascertained by the sub-algorithms 5, 6 at the same time as capture of a second partial dataset 10 of the measurement dataset 3, as FIG. 1 schematically represents by way of the broken-line frame 30. The individual steps and further details of an exemplary advantageous embodiment will be explained in more detail below:

In the exemplary embodiment discussed, the processing facility 20, which implements the ascertainment of the operating parameter 1 as a function of measurement dataset 3, is formed by a first and a second sub-facility 11, 13, which communicate with each other via a bus or a network. In the example, the first sub-facility 11 serves for controlling the imaging and carries out first data processing of incoming measurement data. If the imaging facility 2 is used, for example following ascertainment of the operating parameter 1, for medical imaging, three-dimensional image data can be reconstructed in the first sub-facility 11 already and the second sub-facility 13 can carry out, for example, data visualization for the user.

In the example, the data is processed in the data processing facility 28 forming the first sub-facility 11 by way of a processor 26 a graphics card, with the instructions for a corresponding in data processing being in the form of a computer program 22 in the memory 24 of the first sub-facility 11.

The second sub-facility can be implemented by a data processing facility 29, which is, for example, a workstation computer or a server, with a processor 27 likewise executing a computer program 23 from a memory 25 in order to implement the method steps carried out on the second sub-facility 13. Such two-stage control and data processing is quite usual in the field of medical imaging and will therefore also be used in the example shown. The advantages of this approach have already been stated in the general part.

In the example, an ascertainment instruction 17 is firstly sent in step S1 to the second sub-facility 13 by the first sub-facility 11, receipt of which triggers the capture of the measurement dataset 3 in steps S2 and S3 as well as data processing and transfer by the first sub-facility 11 in the steps S4-S8. Time losses can be avoided and resources of the first sub-facility 11 more efficiently utilized by the use of a joint ascertainment instruction 17 for triggering the capture of measurement data and the evaluation of measurement data, as has already been explained in the general part.

In step S2, firstly only a first partial dataset 9 of the measurement dataset 3 to be captured is captured.

A second partial dataset 10 of the measurement dataset 3 is then subsequently captured in step S3, with steps S4 to S6 also being executed, as indicated by the broken-line frame 30 in FIG. 1, at the same time as this step.

In the example, the partial datasets 9,10 can be assigned to different capture angles and thus comprise, for example, at least one projection image of the X-ray imaging respectively.

In steps S4 and S5, a respective intermediate result 7, 8 is ascertained by a respective sub-algorithm 5, 6 of the processing algorithm 4 on the basis of the first partial dataset 9. By way of example, the sub-algorithm 5 can carry out a summation of the measurement data or the image points in the first partial dataset 9, which can be used for the averaging, which takes place later in step S7, over the two partial datasets 9, 10.

Likewise by way of example, the sub-algorithm 6 can ascertain the ascertainment of a standard deviation within the first partial dataset, for example within a respective projection image, as an intermediate result 8.

As already discussed in the general part, a large number of further sub-algorithms or intermediate results can be used, which could likewise be ascertained in modifications of the example explained, in addition or as an alternative to the intermediate results 7, 8, likewise at the same time as step S3.

In step S6, likewise still at the same time as or overlapping time-wise with the capture of the second partial dataset 10 therefore, the intermediate result 8, which in the example is not processed further in the first sub-facility 11, can be transferred to the second sub-facility 13. Ascertainment of the operating parameter 1 can potentially be accelerated further by the simultaneous implementation of the data transfer for capture of the second partial dataset 10.

As already mentioned, a mean of the measurement data, for example the captured X-ray intensities, over the entire measurement dataset 3 is to be ascertained in step S7 as an example of a processing result 12. Since the sum of the measurement data of the first partial dataset 9 already exists as the intermediate result 7, only a summation of the measurement data of the second partial dataset 10 is still necessary in step S7 and this sum can be added to the intermediate result 7 and then a division carried out to ascertain the mean. Thus, while in the example the entire calculation cannot be carried out before conclusion of the capture of measurement data, a significant portion of the calculation can be brought forward, however, and thus the total time for ascertaining the operating parameter 31 can be reduced in comparison with the customary procedure, carrying out all calculations on completion of measuring therefore.

In step S8, the intermediate result 12 ascertained in step S7 is then transmitted to the first sub-facility 13 in which the operating parameter 1 is ascertained in step S9 by way of a further sub-algorithm 21 of the processing algorithm 4. By way of example, on the basis of the mean ascertained in step S7 and the standard deviation known from step S5, at least one suitable threshold could be selected as the operating parameter 1 for single photon detection and/or a scaling factors could be determined for captured X-ray intensities.

The operating parameter 1 ascertained in step S9 is then transferred in step S10 back to the first sub-facility 11 in order to be used there in subsequent imaging 14. The subsequent imaging 14 can be an optional part of the discussed method, although the method can also end with step S10, and this is indicated by the broken-line arrow in FIG. 1.

The imaging took place in the example in that an image capture command 31 is transferred in step S11 to the first sub-facility 11 by the second sub-facility 13, for example in response to a user input, whereupon this controls the imaging 14 in step S12. The raw data 15 captured in this connection is then processed as a function of the operating parameter 1, in particular in order to provide reconstructed image data.

For reasons of clarity, it will be assumed in the exemplary embodiment shown in FIG. 1 that the measurement dataset 3 comprises only two successively captured partial datasets 9,10. In real implementations of the method, it is typically advantageous to use a higher number of partial datasets, with it being possible to ascertain, in particular during the capture of a respective subsequently captured one of the partial datasets, intermediate results on the basis of any previously captured partial dataset or a plurality of the previously captured partial datasets.

If, when using a plurality of partial datasets, a mean of all measurement data, for example, is then to be ascertained, as has already been explained with reference to steps S4 and S7 for the case of two partial datasets 9,10, the summation can take place at the same time as capture of measurement data for all partial datasets except for the most recently captured of the partial datasets, so nearly all of the computing time for summation can be expended during the measuring process. This results in a significant reduction in the time necessary for ascertaining the operating parameter.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

Independent of the grammatical term usage, individuals with male, female or other gender identities are included within the term.

What is claimed is:

1. A method for ascertaining an operating parameter of a medical imaging device, the method comprising:
providing a measurement dataset by operation of the medical imaging device; and
ascertaining the operating parameter as a function of the measurement dataset, by a processing algorithm, wherein
the processing algorithm includes a sub-algorithm providing an intermediate result,
the intermediate result depends solely on a first partial dataset of the measurement dataset,
the operating parameter is ascertained as a function of the intermediate result and a second partial dataset of the measurement dataset, and
calculation of the intermediate result by the sub-algorithm and capture of the second partial dataset by the medical imaging device at least partially overlap time-wise.

2. The method as claimed in claim 1, wherein
the sub-algorithm is implemented by a first sub-device,
the first sub-device transfers, to a second sub-device, at least one of the intermediate result or a processing result ascertained as a function of the intermediate result, and
the operating parameter is ascertained by the second sub-device.

3. The method as claimed in claim 2, wherein transfer of at least one of the intermediate result or the processing result to the second sub-device and the capture of the second partial dataset by the medical imaging device at least partially overlap time-wise.

4. The method as claimed in claim 2, wherein after ascertaining the operating parameter, the method further comprises:
transferring the operating parameter from the second sub-device to the first sub-device; and
storing the operating parameter at the first sub-device in order for the first sub-device to use the operating parameter during subsequent imaging by the medical imaging device for at least one of processing captured raw data or controlling at least one component of the medical imaging device.

5. The method as claimed in claim 2, wherein capture of the measurement dataset and at least execution of the sub-algorithm is triggered by the first sub-device in response to an ascertainment instruction from the second sub-device.

6. The method as claimed in claim 1, wherein
the measurement dataset includes measurement data for a plurality of capture angles, and
the first partial dataset includes measurement data for one more of the plurality of capture angles.

7. The method as claimed in claim 1, wherein the sub-algorithm comprises at least one of
at least one of a summation or a logarithmizing of measurement data of the first partial dataset or preprocessed data ascertained from the first partial dataset,
an assignment of value to the measurement data or to the preprocessed data with the aid of a lookup table,
a statistical evaluation of the measurement data or the preprocessed data, wherein the statistical evaluation includes at least one of an averaging or ascertainment of a standard deviation, or
ascertainment of a noise figure relating to a noise component in the measurement data.

8. The method as claimed in claim 1, wherein at least one of
a respective operating parameter is captured by a plurality of processing algorithms, wherein the plurality of processing algorithms includes at least one parallelizable sub-algorithm respectively, or
the processing algorithm includes a plurality of parallelizable sub-algorithms, wherein
the plurality of parallelizable sub-algorithms process at least one of the first partial dataset or a further partial dataset respectively, which is captured before the second partial dataset, in order to ascertain a respective intermediate result, wherein calculations of the respective intermediate results by the respective parallelizable sub-algorithms at least partially overlap time-wise with one another and with the capture of the second partial dataset by the medical imaging device.

9. The method as claimed in claim 1, further comprising:
ascertaining, as the operating parameter, at least one of a measure of a sensitivity of detector elements of the medical imaging device, at least one threshold value for single photon detection, at least one scaling factor used by the medical imaging device during imaging, at least one entry of a lookup table used during imaging, or at least one parameter of a filter function used during imaging.

10. The method as claimed in claim 1, wherein the medical imaging device is a computed tomography device, a device for molecular imaging or a magnetic resonance tomograph.

11. A medical imaging device, comprising:
at least one detector configured to capture a measurement dataset; and
a processing device configured to carry out the method as claimed in claim 1.

12. The medical imaging device as claimed in claim 11, wherein the processing device comprises:
a first sub-device and a second sub-device, wherein
the first sub-device is configured to implement the sub-algorithm, and
transfer, to the second sub-device, at least one of the intermediate result or a processing result ascertained as a function of the intermediate result, and
the second sub-device is configured to ascertain the operating parameter as a function of the at least one of the intermediate result or the processing result.

13. A non-transitory computer-readable storage medium storing computer-executable instructions that, when executed at a processor of a medical imaging device, cause the medical imaging device to perform the method of claim 1.

14. The method as claimed in claim 3, wherein after ascertaining the operating parameter, the method further comprises:
transferring the operating parameter from the second sub-device to the first sub-device; and
storing the operating parameter at the first sub-device in order for the first sub-device to use the operating parameter during subsequent imaging by the medical imaging device for at least one of processing captured raw data or controlling at least one component of the medical imaging device.

15. The method as claimed in claim 14, wherein capture of the measurement dataset and at least execution of the sub-algorithm is triggered by the first sub-device in response to an ascertainment instruction from the second sub-device.

16. The method as claimed in claim 2, wherein
the measurement dataset includes measurement data for a plurality of capture angles, and
the first partial dataset includes measurement data for one more of the plurality of capture angles.

17. The method as claimed in claim 4, further comprising:
ascertaining, as the operating parameter, at least one of a measure of a sensitivity of detector elements of the medical imaging device, at least one threshold value for single photon detection, at least one scaling factor used by the medical imaging device during imaging, at least one entry of a lookup table used during imaging, or at least one parameter of a filter function used during imaging.

18. The method as claimed in claim 5, further comprising:
ascertaining, as the operating parameter, at least one of a measure of a sensitivity of detector elements of the medical imaging device, at least one threshold value for single photon detection, at least one scaling factor used by the medical imaging device during imaging, at least one entry of a lookup table used during imaging, or at least one parameter of a filter function used during imaging.

19. The method as claimed in claim 1, wherein the medical imaging device is an x-ray device.

20. A medical imaging device, comprising:
a memory storing computer-readable instructions; and
at least one processor configured to execute the computer-readable instructions to cause the medical imaging device to
ascertain an operating parameter of the medical imaging device as a function of a measurement dataset by a processing algorithm, wherein
the processing algorithm includes a sub-algorithm providing an intermediate result,
the intermediate result depends on a first partial dataset of the measurement dataset,
the operating parameter is ascertained as a function of the intermediate result and a second partial dataset of the measurement dataset, and
calculation of the intermediate result by the sub-algorithm and capture of the second partial dataset by the medical imaging device at least partially overlap time-wise.
